(19) United States
(12) Patent Application Publication
(10) Pub. No.: US 2024/0091189 A1
(43) Pub. Date: Mar. 21, 2024

(54) Methods for Assessing a Patient's Response to Treatment of a Neurodegenerative Disease with Deuterated Arachidonic Acid

(71) Applicant: Retrotope, Inc., Los Altos, CA (US)

(72) Inventors: Peter Milner, Los Altos, CA (US); Mikhail Sergeevich Shchepinov, Oxford (GB)

(21) Appl. No.: 18/275,968

(22) PCT Filed: Feb. 4, 2022
(86) PCT No.: PCT/US2022/015368

§ 371 Date: Aug. 4, 2023

Related U.S. Application Data
(63) Continuation of application No. 17/391,909, filed on Aug. 2, 2021, now Pat. No. 11,491,130.
Application No. 17/391,909 is a continuation-in-part of application No. 17/169,271, filed on Feb. 5, 2021, now Pat. No. 11,351,143.
(60) Provisional application No. 63/177,794, filed on Apr. 21, 2021.

(51) Int. Cl. A61K 31/202; A61K 31/232; A61P 25/28

(52) U.S. Cl. CPC A61K 31/202; A61K 31/232; A61P 25/28

(57) ABSTRACT

Disclosed are diagnostic methods for assessing the presence or absence of a therapeutic concentration of a deuterated polyunsaturated fatty acid in diseased neurons during treatment of a patient with a neurodegenerative disease.

### CROSS-REFERENCE TO RELATED APPLICATIONS

This application claims priority to U.S. patent application Ser. No. 17/169,271, filed on Feb. 5, 2021; U.S. patent application Ser. No. 17/391,909, filed Aug. 2, 2021; and U.S. Provisional Patent Application No. 63/177,794, filed Apr. 21, 2021, each of which is incorporated herein by reference in its entirety.

### FIELD OF THE INVENTION

Disclosed are diagnostic methods for assessing the presence or absence of a therapeutic concentration of a deuterated polyunsaturated fatty acid in diseased neurons during treatment of a patient with a neurodegenerative disease.

### STATE OF THE ART

Lipid auto-oxidation of polyunsaturated fatty acids (PUFAs) in neurons is associated with the pathology of numerous neurodegenerative diseases. Central to this oxidative pathway is the presence of labile bis-allylic hydrogen atoms found in arachidonic acid, the dominant PUFA found in neurons.

In cellular membranes, arachidonic acids are stacked together and oxidative processes involving reactive oxygen species (ROS) act as an initiator for autoxidation of these PUFAs by extraction of the bis-allylic hydrogen and formation of an oxidative reactive species in the PUFA. Initial oxidation at a first bis-allylic site then leads to serial oxidation of further PUFAs in the membrane of the cell or the mitochondria. The oxidative process starts with hydrogen extraction at a bis-allylic site on the first PUFA and proceeds in a serial manner to the next PUFA and then the next PUFA and so on. At some point the oxidative process damages or destroys the viability of the neuron leading to furtherance of the disease condition that is responsible for generation of the excessive amounts of ROS.

Heretofore, the art has disclosed that the progression of neurodegenerative diseases can be attenuated by deuteration at one or more of the bis-allylic sites of arachidonic acid found in the neurons. The stability of the deuterium-carbon bond against such oxidative processes is significantly stronger (more stable) than that of the hydrogen-carbon bond. This means that the generation of an oxidative species at the bis-allylic sites is so reduced by the carbon-deuterium bonds that the auto-oxidative pathway is inhibited. In turn, termination of this pathway leads to enhanced survival of the neurons and, as such, attenuates the progression of the disease.

Clinical studies to date have shown that certain neurodegenerative diseases are responsive to such therapy including tauopathy, amyotrophic lateral sclerosis (ALS), Infantile Neuroaxonal Dystrophy (INAD), and Friedreich Ataxia (FA). In addition, preclinical studies evidence that certain other neurodegenerative diseases are similarly responsive to such therapy including mitochondrial deficiencies involving ROS, Alzheimer's Disease, Huntington's Disease, Parkinson's Disease, schizophrenia and bipolar disorder, just to name a few.

Treatment of these neurodegenerative diseases with deuterated PUFAs is typically conducted by administration of the active deuterated PUFA or a prodrug thereof. For example, in neurodegenerative diseases, the active deuterated PUFA is deuterated arachidonic acid. However, one can treat this disease by administering either a deuterated arachidonic acid or a prodrug thereof. In one case, the prodrug is a C₁-C₅ alkyl ester of a deuterated arachidonic acid. In another case, the prodrug is a deuterated linoleic acid or a C₁-C₅ alkyl ester thereof. In the case of esters of either of these PUFAs, they are readily removed by the conditions found in the stomach to provide for the free acid or a salt thereof. In the case of deuterated linoleic acid, a portion of this compound is enzymatically converted to deuterated arachidonic acid thereby providing a source of deuterated arachidonic acid in the patient.

While this therapy has been shown to be successful in clinic studies as well as in animal models, there is a need to determine whether a given patient is achieving or maintaining a therapeutic concentration of the deuterated arachidonic acid in his/her neurons. This concern arises from a variety of difficult to control complications. These include the following:
PUFAs incorporated into cellular membranes, including arachidonic acid, have a half-life dictated by their turnover rate—the rate a damaged PUFA is removed and replaced by a new PUFA coupled with the rate of extraction of arachidonic acid molecules from the cell membrane as part of bioconversion to prostaglandins, prostacyclin (PGI₂), thromboxane A₂ (TxA₂), or a series of hydroxyeicosatetraenoic acids (HETEs), epoxyeicosatrienoic acids (IEM's), and di hydroxyeicosatrienoic acids (DiHETEs) as well as multiple other catabolic products;
the amount of PUFAs (including deuterated PUFAs) absorbed by a person is dependent on their total consumption of PUFAs. Typically, a person's metabolism is not capable of absorbing all of the PUFAs consumed with the percentage absorbed PUFAs diminishing as the amount of PUFAs consumed increases. Since the deuterated PUFAs are included in the total amount of PUFAs consumed, the percentage of non-absorbed PUFAs will include the same percentage of non-absorbed deuterated PUFAs. So, if only 80% of the total PUFAs consumed is absorbed, then only 80% of the deuterated PUFA is absorbed. Still further, the amount absorbed of course is a variable that changes from individual to individual;
the amount of PUFA absorbed is also dependent on whether such absorption is interfered with a digestive problem such as diarrhea associated with C. difficile. In such cases, many nutrients including consumed PUFAs are not absorbed into the body but are merely “washed out”;
even in a given individual, their diet is not stagnant. So, over time, the amount of PUFAs consumed can change as will the percentage absorbed; and
finally, the question of patient compliance with his/her dosing of deuterated PUFAs is a relevant factor relating to the amount of deuterated PUFAs absorbed.

All of these concerns dictate that patients undergoing deuterated PUFA therapy should be evaluated to determine whether they are achieving and/or maintaining a therapeutic concentration in their neurons.

The evaluation of the concentration of deuterated arachidonic acid in the neurons is further complicated because the neurons are inaccessible and the clinician cannot merely extract a sample of these neurons to evaluate the concentration of deuterated arachidonic acid contained therein. While the administered deuterated arachidonic acid is systemically incorporated into all cells, the data to date suggests that the body shunts a larger portion of deuterated arachidonic acid to the neurons and the spinal fluid as compared to other parts of the body. Thus, the concentration of deuterated arachidonic acid in a surrogate or reporter cell such as a skin cell, a red blood cell, etc. does not necessarily represent the concentration of deuterated arachidonic acid in the neurons or in the cerebral spinal fluid. Moreover, spinal taps to extract cerebral spinal fluid are not feasible as spinal taps involve a significant amount of time (e.g., 30 minutes or so) and can cause significant discomfort to the patient including headaches that last for days or weeks and back pain.

Accordingly, there is an ongoing need for safe and effective diagnostic tests that can determine if a patient having a neurodegenerative disease treatable with a deuterated PUFA has a therapeutic or sub-therapeutic concentration of that deuterated arachidonic acid in his/her neurons. In addition, it is helpful in evaluating whether the patient's metabolism evidences appropriate absorption of the deuterated arachidonic acid.

### SUMMARY

Provided herein is a diagnostic test for accessing whether a therapeutic concentration of deuterated arachidonic acid is present in the neurons of patients being treated for a neurodegenerative disease treatable with deuterated arachidonic acid or a prodrug thereof without the need to access the patient's neurons or cerebral spinal fluid. This test is based, in part, on defining a reproducible correlation from different clinical studies treating different treatable neurodegenerative diseases all of which use the same deuterated arachidonic acid, an ester thereof or a prodrug thereof.

The amount of deuterated arachidonic acid incorporated into cells incrementally increases over time. As such, a therapeutic concentration of the deuterated arachidonic acid is not immediately achieved. Rather, there is lag time between initiation of therapy and onset of a therapeutic result that can vary from patient to patient. At some point after the start of therapy for a neurodegenerative disease, one or more evaluations of the patient's physical response to that therapy is (are) required to determine when a therapeutic result is achieved and whether the therapeutic result is being maintained. In one embodiment, such physical evaluations are coupled with obtaining a sample of the patient's reporter cells that are then used to determine the concentration of deuterated arachidonic acid in said cells. Alternatively, samples of the patient's reporter cells are obtained at defined intervals. When a therapeutic result in the patient is first confirmed, the clinician or statistician can correlate the concentration of the deuterated arachidonic acid in the reporter cells at that time as the therapeutic required minimum concentration.

When the therapeutic concentration from clinical studies for different neurodegenerative diseases was evaluated, the data supported the conclusion that there was an equivalent therapeutic concentration of deuterated arachidonic acid that provided for therapeutic results for each of these diseases. Since each of these diseases involves a different etiology, the fact that equivalent therapeutic concentrations of deuterated arachidonic acid were found to be therapeutic was surprising.

Accordingly, in one embodiment, there is provided a method for determining a minimum therapeutic concentration of deuterated arachidonic acid in neurons of patients suffering from different neurodegenerative diseases treatable with deuterated arachidonic acid without accessing either the neurons or the cerebral spinal fluid of said patients, which method comprises:
a) obtaining data from human clinical studies for a representative number of neurodegenerative diseases each treatable with the same deuterated arachidonic acid or a prodrug thereof which data includes the concentration of deuterated arachidonic acid in an accessible reporter cell population at one or more times after the start of therapy;
b) assessing when the patients evidence onset of a therapeutic result;
c) identifying the average time from initiation of therapy to onset of therapeutic results and the average concentration of deuterated arachidonic acid in the reporter cells at that time of such onset for each neurodegenerative disease;
d) correlating the average concentration of the deuterated arachidonic acid in said population of reporter cells to the average time for the onset of a minimum required therapeutic result for each neurodegenerative disease as evidencing that said neurons have a minimum required therapeutic concentration of the deuterated arachidonic acid for that disease;
e) confirming that said minimum required therapeutic concentration of deuterated arachidonic acid and said average time for onset of said therapeutic results for each neurodegenerative disease is equivalent to the other neurodegenerative diseases; and
f) based on said equivalence ascribe said average minimum required therapeutic concentration and said average time for the onset of said therapeutic results to other neurodegenerative diseases.

In some embodiments, there is provided a non-invasive method for determining a concentration of deuterated arachidonic acid in reporter cells of a population of patients that correlates to onset of a therapeutic effect in a patient population suffering from a neurodegenerative disease treatable with said deuterated arachidonic acid, the method comprising:
a) obtaining data from the population of patients having a first neurodegenerative disease during treatment regimen with deuterated arachidonic acid or a prodrug thereof, which data comprises the concentration of deuterated arachidonic acid in the population of patients' reporter cells at one or more times after the start of therapy;
b) assessing when the patients evidence a therapeutic result;
c) identifying the average time from initiation of therapy to onset of therapeutic results and the average concentration of deuterated arachidonic acid in the reporter cells of each patient at that time of such onset for each neurodegenerative disease;
d) correlating the average concentration of the deuterated arachidonic acid in said population of patients' reporter cells to the average time for the onset of a minimum required therapeutic result for said neurodegenerative disease as evidencing that patient neurons have a minimum required therapeutic concentration of the deuterated arachidonic acid for that disease;
e) repeating steps a)-d) for one or more further neurodegenerative diseases;
f) evaluating a minimum required therapeutic concentration of deuterated arachidonic acid and average time for onset of therapeutic results for each of said one or more further neurodegenerative diseases; and
f) obtaining an average minimum required therapeutic concentration and average time for the onset of said therapeutic results for a set of neurodegenerative diseases.

In some embodiments, there is provided a method for determining the response of a patient to the administration of a composition comprising deuterated arachidonic acid or a prodrug thereof during the treatment for a treatable neurodegenerative disease, said method comprises:
obtaining a population of red blood cells from said patient suffering from a neurodegenerative disease at a set point in time after the start of therapy predetermined to be when a therapeutic result should be evident for that disease;
assessing the concentration of said deuterated arachidonic acid in said red blood cells at that time wherein said concentration is relative to the amount of arachidonic acid present including deuterated arachidonic acid;
comparing said assessed concentration to said predetermined concentration as to when a therapeutic result is evident;
determining if said assessed concentration is therapeutic or sub-therapeutic for treatable neurodegenerative diseases; and
optionally altering the dose of said deuterated arachidonic acid or prodrug thereof to said patient based on the said determination.

In one embodiment, there is provided a method for determining whether a patient undergoing treatment for a treatable neurodegenerative disease with a deuterated arachidonic acid or a prodrug thereof has achieved onset of therapy or is maintaining a therapeutic concentration of said deuterated arachidonic acid in the patient's neurons said method comprises:
obtaining a population of reporter cells from said patient suffering from a neurodegenerative disease at a set point in time after the start of therapy predetermined to be when a therapeutic result should be evident for that disease;
assessing the concentration of said deuterated arachidonic acid in said reporter cells at that time;
comparing said assessed concentration to said predetermined concentration as to when a therapeutic result is evident;
determining if said assessed concentration is therapeutic or sub-therapeutic for treatable neurodegenerative diseases; and
optionally altering the dose of said deuterated arachidonic acid or prodrug thereof to said patient based on the said determination.

In one embodiment, the method is repeated at later times to assess whether the patient is achieving or maintaining a therapeutic concentration.

In one embodiment, the sample of reporter cells is obtained at a set period of time that is about 1 month after the start of therapy. Optional subsequent samples can be obtained at intervals such as 1 month thereafter, 3 months thereafter, semi-annually, or annually.

In one embodiment, the deuterated arachidonic acid is D2-arachidonic acid, D4-arachidonic acid or D6-arachidonic acid. Non-limiting examples of D2-arachidonic acid include 7,7-D2-arachidonic acid, 10,10-D2-arachidonic acid and 13,13-D2-arachidonic acid. Non-limiting examples of D4-arachidonic acid include 7,7,10,10-arachidonic acid, 7,7,13,13-D4 arachidonic acid, 10,10,13,13-D4-arachidonic acid. D6-arachidonic acid (as defined herein) includes 7,7,10,10,13,13-D6-arachidonic acid. Preferably, the deuterated arachidonic acid is D2-arachidonic acid or D6-arachidonic acid. In some embodiments, D6-arachidonic acid comprises both 7,7,10,10,13,13-D6-arachidonic acid as well as a composition comprising deuterated arachidonic acid wherein at least 90% of the hydrogen atoms at the 7,7,10,10,13,13 positions are replaced with deuterium and having at least one non-bis-allylic position having a level of deuterium substitution above its natural abundance. In one embodiment, the non-bis-allylic positions optionally have up to about 35% of the remaining hydrogen atoms replaced by deuterium and, preferably, from about 1 to 35% and, more preferably, from about 1 to 10%.

In one embodiment, the reporter cells are any accessible cells in the body and, preferably, are readily accessible without causing the patient undue pain or inconvenience. Suitable reporter cells include by way of example only red blood cells, skin cells, fat cells, biopsied cells, epithelial cells including those found in urine, and the like. In one embodiment, the reporter cells are preferably red blood cells.

In one embodiment, there is provided a method for determining the response of a patient to the administration of a composition comprising deuterated arachidonic acid or a prodrug thereof during the treatment for a treatable neurodegenerative disease, said method comprises:
obtaining a population of red blood cells from said patient suffering from a neurodegenerative disease at a set point in time after the start of therapy predetermined to be when a therapeutic result should be evident for that disease;
assessing the concentration of said deuterated arachidonic acid in said red blood cells at that time;
comparing said assessed concentration to said predetermined concentration as to when a therapeutic result is evident;
determining if said assessed concentration is therapeutic or sub-therapeutic for treatable neurodegenerative diseases; and
optionally altering the dose of said deuterated arachidonic acid or prodrug thereof to said patient based on the said determination.

In one embodiment, separate clinical studies of treatable neurodegenerative diseases evidence that a concentration of deuterated 13,13-D2-arachidonic acid of about 3% or more relative to the total amount of arachidonic acid including deuterated arachidonic acid), in red blood cells acting as the reporter cell, correlates to the minimum concentration needed to achieve therapeutic results in these neurodegenerative diseases. As such, this concentration establishes the baseline for achieving therapeutic results in those neurodegenerative diseases treatable with 13,13-D2-arachidonic acid and evidence that the 13,13-D2-arachidonic acid concentration in the diseased neurons is sufficient to provide therapy for the treated disease.

In one embodiment, this invention provides for a diagnostic test to determine whether the concentration of deuterated 13,13-D2-arachidonic acid in a patient with a treatable neurodegenerative disease is therapeutic or sub-therapeutic without accessing the patient's neurons or cerebral spinal fluid, which test comprises:
obtaining a blood sample from a patient being treated for a neurodegenerative disease with 13,13-D2-arachidonic acid or an ester thereof;
determining the concentration of 13,13-D2-arachidonic acid in red blood cells (RBCs); and
comparing that concentration to a minimum therapeutic concentration of least 3% of 13,13-D2-arachidonic acid based on the total amount of arachidonic acid including deuterated arachidonic acid in said RBCs to determine if the patient has a therapeutic concentration or a sub-therapeutic of D2-arachidonic acid in the neurons.

In one embodiment, higher concentrations of 13,13-D2-arachidonic acid are contemplated as providing for enhanced therapeutic results and the attending clinician may conclude that such higher concentrations should be the target concentrations for a therapeutic result for a particular patient or group of patients. For example, concentrations of 13,13-D2-arachidonic acid may be set at 4%, 5%, 6%, 7%, 8%, 9% or even 10% as the particular therapeutic target for a given patient recognizing that a concentration of 13,13-D2-arachidonic acid is the baseline for a minimum required therapeutic result. Increasing the target concentration by the attending clinician can be based on factors such as the age and weight of the patient, the condition of the patient, the progression of the disease, as well as other factors well known to the clinician.

The minimum required therapeutic concentration of D2-arachidonic acid provided above can be used to correlate the minimum therapeutic concentration of D4-arachidonic acid or D6-arachidonic acid recognizing that deuteration at one or both of the remaining bis-allylic sites increases the stability of arachidonic acid against lipid autoxidation.

In one embodiment, there is provided a diagnostic test to determine whether the concentration of deuterated D4-arachidonic acid in a patient with a treatable neurodegenerative disease is therapeutic or sub-therapeutic without accessing the patient's neurons or cerebral spinal fluid, which test comprises:
obtaining a blood sample from a patient being treated for a neurodegenerative disease with D4-arachidonic acid or an ester thereof;
determining the concentration of D4-arachidonic acid in red blood cells (RBCs); and comparing that concentration to a minimum therapeutic concentration of least 1% of D4-arachidonic acid based on the total amount of arachidonic acid including deuterated arachidonic acid in said RBCs to determine if the patient has a therapeutic concentration or a sub-therapeutic of D4-arachidonic acid in the neurons.

In one embodiment, there is provided a diagnostic test to determine whether the concentration of deuterated D6-arachidonic acid in a patient with a treatable neurodegenerative disease is therapeutic or sub-therapeutic without accessing the patient's neurons or cerebral spinal fluid, which test comprises:
obtaining a blood sample from a patient being treated for a neurodegenerative disease with D4-arachidonic acid or an ester thereof;
determining the concentration of D6-arachidonic acid in red blood cells (RBCs); and comparing that concentration to a minimum therapeutic concentration of least 0.5% of D6-arachidonic acid based on the total amount of arachidonic acid including deuterated arachidonice acid in said RBCs to determine if the patient has a therapeutic concentration or a sub-therapeutic of D6-arachidonic acid in the neurons.

In one embodiment, higher concentrations of either D4- or D6-arachidonic acid provide for enhanced therapeutic results and the attending clinician may conclude that these concentrations are the minimum required target concentrations for a therapeutic result if an enhanced result is set as the target concentration. For example, concentrations of D4- and D6-arachidonic acid may be set at follow Table 1 where concentrations are determined as above:

TABLE 1

| Deuterated | | | More | Most |
|---|---|---|---|---|
| Arachidonic | | Preferred | Preferred | Preferred |
| Acid | Conc. * | Conc. * | Conc. * | Conc. * |
| D4- | at least | at least | at least about | at least |
| arachidonic | about 1% | about 2% | about 3% | about 5% |
| acid | | | | |
| D6- | at least | at least | at least | at least |
| arachidonic | about 0.5% | about 1% | about 1.5% | about 2.5% |
| acid | | | or at least | or at least |
| | | | about 2% | about 3% |

* in red blood cells

In one embodiment, there is provide a kit of parts which includes diagnostic materials for conducting said methods as well as a correlation table that correlates the concentration of the deuterated arachidonic acid in the red blood cells to that of the cerebral spinal fluid in humans. In addition to the correlation table, such diagnostic materials can include one or more instructions for when such tests should be conducted, factors that suggest delaying such tests, and the like.

### BRIEF DESCRIPTION OF THE DRAWINGS

FIG. 1 is a graph showing the percent of 13,13-D2-Arachidonic Acid in red blood cells (RBC) and spinal fluid (SF) at the indicated time points after start of treatment with 11,11-D2-Linoleic Acid in an adult patient.

FIG. 2 is a graph showing the percent of 13,13-D2-Arachidonic Acid in red blood cells (RBC) and spinal fluid (SF) at the indicated time points after start of treatment with 11,11-D2-Linoleic Acid in juvenile patients.

### DETAILED DESCRIPTION

Provided are diagnostic methods or tests for determining whether a patient exhibits a minimum therapeutic concentration of deuterated arachidonic acid in the neurons of a patient being treated for a treatable neurodegenerative disease. Also provided are methods or tests to determine if a patient being treated for a treatable neurodegenerative disease has achieved or is maintaining a therapeutic concentration of the deuterated arachidonic acid.

Prior to discussing this invention in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by plus (+) or minus (−) 10%, 5%, 1%, or any subrange or subvalue there between. Preferably, the term “about” when used with regard to a minimum therapeutic concentration means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, the term “linoleic acid” refers to the compound and a pharmaceutically acceptable salt thereof having the formula provided below and having the natural abundance of deuterium at each hydrogen atom:

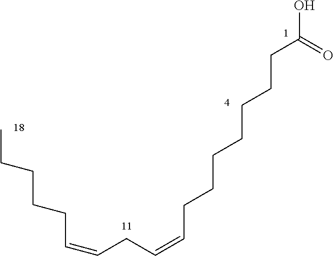 [SMILES: CCCCC/C=C\C/C=C\CCCCCCCC(=O)O]

Esters of linoleic acid are formed by replacing the —OH group with —OR. Such esters are as defined herein below.

As used herein and unless the context dictates otherwise, the term “deuterated linoleic acid or an ester thereof” refers to linoleic acid or ester compounds comprising one or two deuterium atoms at the 11 position thereof and optionally additional deuterium atoms at other positions within the molecule including at position 8. Specific compounds encompassed by this definition include by way of example only 11-D1-linoleic acid, 11,11-D2-linoleic acid, 8,11-D2-linoleic acid, 8,11,11-D3-linoleic acid and 8,8,11,11-D4-linoleic acid as well as esters of any one of these compounds. Additional stabilization of the bis-allylic position could also include replacement of one or more of bis-allylic carbon atoms with a heavy isotope, alone or in conjunction with the deuteration (or tritiation), as the isotope effect (IE) resulting in stabilization of a bond with heavy isotopes is additive per long-established and fundamental chemical principles. (Westheimer, Chem. Rev. (1961), 61:265-273; Shchepinov, Rejuvenation Res., (2007), 10:47-59; Hill et al., Free Radic. Biol. Med., (2012), 53:893-906; Andreyev et al., Free Radic. Biol. Med., (2015), 82:63-72. Bigeleisen, J. The validity of the use of tracers to follow chemical reactions. Science, (1949), 110:14-16.

As used herein, arachidonic acid has the numbering system as described below:

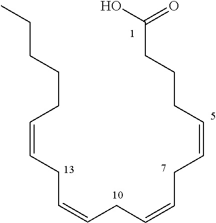 [SMILES: CCCCC/C=C\C/C=C\C/C=C\C/C=C\CCCC(=O)O]

where each of positions 7, 10 and 13 are bis-allylic positions within the structure.

As used herein and unless the context dictates otherwise, the term “deuterated arachidonic acid or an ester thereof” refers to arachidonic acid or ester compounds having at least one deuterium atom at a bis-allylic position and optionally additional deuterium atoms at other positions within the molecule. Such deuterated arachidonic acids include mono-bis-allylic deuterated arachidonic acid having a deuterium atom at the 7, 10 or 13 position as well di-, tri-, tetra-, penta-, hexa-deuterated at the bis-allylic sites and optionally deuteration at sites other than the bis-allylic sites at a level greater than the natural abundance of deuterium.

The term deuterated arachidonic acid includes 7,7-D2-arachidonic acid, 10,10-D2-arachidonic acid, acid, and 13,13-D2-arachidonic acid. The term D4-arachidonic acid includes 7,7,10,10-D4-arachidonic acid, 7,7,13,13-D4-arachidonic acid, 10,10,13,13-D4-arachidonic acid. The term D6-arachidonic acid includes 7,7,10,10,13,13-D6-arachidonic acid as well as deuterated arachidonic acid having at least 90% of the hydrogen atoms at the 7,7,10,10,13,13 positions replaced with deuterium and optionally having up to 35% of the remaining hydrogen atoms at the non-bis-allylic sites replaced by deuterium.

As used herein, the term “ester” means any pharmaceutically acceptable ester of a deuterated linoleic acid or a deuterated arachidonic acid such as but not limited to C₁-C₆ alkyl esters, glycerol (including monoglycerides, diglycerides and triglycerides), sucrose esters, phosphate esters, and the like. The particular ester employed is not critical provided that the ester is pharmaceutically acceptable (non-toxic and biocompatible).

As used herein, the term “prodrug of deuterated arachidonic acid” refers to esters of deuterated arachidonic acid such as C₁-C₅ alkyl esters, deuterated linoleic acid, and esters of deuterated linoleic acid such as the C₁-C₅ alkyl esters. Esters of both deuterated arachidonic acid and deuterated linoleic acid are readily converted into their corresponding acid/salt form in the gastro-intestinal track after administration. A portion of the administered deuterated linoleic acid is enzymatically converted to deuterated arachidonic acid and, as such, act as a prodrug for such deuterated arachidonic acids. In one embodiment, 11,11-D2-linoleic acid is converted to 13,13-D2-arachidonic acid. In one embodiment, 8,8,11,11-D4-linoleic acid is converted to 10,10,13,13-D4-arachidonic acid.

The term “treatable neurodegenerative disease” or “neurodegenerative disease treatable with a deuterated arachidonic acid” means that the specific neurodegenerative disease is recognized to be treatable with deuterated arachidonic acid as compared to neurodegenerative diseases that are not treatable with deuterated arachidonic acid. Neurodegenerative diseases treatable with deuterated arachidonic acid include Alzheimer's Disease, mild cognitive impairment, frontotemporal dementia, amyotrophic lateral sclerosis, multiple sclerosis, Friedreich's ataxia, Parkinson's disease, tauopathy (including PSP), and Huntington's disease. On the other hand, the data to date suggest that the following diseases are not treatable with deuterated arachidonic acid and/or other deuterated polyunsaturated fatty acids including Tay-Sach's Disease, GPX-4 deficiency, and neuroserpinosis.

As used herein, the term “etiology” of a disease refers to the cause of that disease. The term “pathogenesis” or “pathology” refers to the development, structural/functional changes, and natural history associated with that disease. The term “natural history” means the progression of the disease in the absence of treatment or the in the absence of treatment using deuterated arachidonic acid or a prodrug thereof.

As used herein, the term “reduced rate of disease progression” means that the rate of disease progression is attenuated after initiation of treatment as compared to the patient's natural history. In one embodiment, the reduced rate of disease progression in ALS is measured by using the ALSFRS-R score to determine the rate of disease progression during the natural history and, again, measuring the ALSFRS-R score during the interval starting with therapy and ending at a set period of time thereafter (e.g., 6 months). Both rates are then annualized and a reduced rate of disease progression results in a percentage change of at least 30% between the ALSFRS-R scores before and after.

For PSP, the rate of disease progression is measured by using the Progressive Supranuclear Palsy Rating Scale or the Unified Parkinson's Disease Rating Scale to determine the rate of disease progression during the natural history and, again, measuring either score during the interval starting with therapy and again after a set period of time thereafter (e.g., at 1 month and at every 3 months thereafter). Both rates are then annualized and a reduced rate of disease progression results in a percentage change of at least 30% in the after score as compared to the before score.

Likewise, there are similar standardized scales to measure the rate of disease progression in Alzheimer's disease, Parkinson's disease, Huntington's disease, Friedreich's ataxia, and the like.

As used herein, the term a “minimum required therapeutic result” means when a patient being treated with a deuterated arachidonic acid or prodrug thereof evidences a rate of reduction in disease progression of at least about 30% as compared to the rate of disease progression determined during the natural history.

As used herein, the term “patient” refers to a human patient or a cohort of human patients suffering from a neurodegenerative disease. When more than 1 patient is evaluated, then the average of their disease progression is used.

As used herein, the term “pharmaceutically acceptable salts” of compounds disclosed herein are within the scope of the present invention include acid or base addition salts which retain the desired pharmacological activity and is not biologically undesirable (e.g., the salt is not unduly toxic, allergenic, or irritating, and is bioavailable). When the compound of the present invention has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present invention has an acidic group, such as for example, a carboxylic acid group, it can form salts with metals, such as alkali and earth alkali metals (e.g., Na⁺, K⁺, Ca⁺, Mg⁺, Zn⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

### Compounds

A portion of the deuterated linoleic acid or an ester thereof administered to a patient is converted as the de-esterified acid or salt thereof into deuterated arachidonic acid in vivo and, that portion of the deuterated linoleic acid so converted acts as a prodrug of deuterated arachidonic acid. There are numerous deuterated linoleic acid compounds that are commercially available or known in the art. These include, by way of example only, 11-D1-linoleic acid, 11,11-D2-linoleic acid, 8,8,11,11-D4 linoleic acid (which yields 10,10,13,13-D4-arachidonic acid upon enzymatic desaturation-extension). Other deuterated linoleic acids are described in U.S. Pat. No. 10,052,299 which is incorporated herein by reference in its entirety. In addition, 11-D1-linoleic acid is commercially available from Cayman Chemical Company, Ann Arbor, Michigan, USA 48108.

In addition to deuterated linoleic acid or an ester thereof, one can bypass the enzymatic conversion required for that prodrug by administering deuterated arachidonic acid to the patient. Such deuterated arachidonic acids include 7,7-D2-arachidonic acid, 10,10-D2-arachidonic acid, 13,13-D2-arachidonic acid, 7,7,10,10-D4-arachidonic acid, 7,7,13,13-D4 arachidonic acid, 10,10,13,13-D4-arachidonic acid, 7,7,10,10,13,13-D6-arachidonic acid are disclosed by Shchepinov, et al., Molecules, 28(12):3331 et seq. (2018) which is incorporated herein by reference in its entirety. Other deuterated arachidonic acid compounds are known in the art.

Still further, deuterated arachidonic acids can be prepare by catalytic processes as described in U.S. Pat. No. 10,577,304 which patent is incorporated herein by reference in its entirety. Those processes provide for substantially complete deuteration at the bis-allylic sites with some deuteration at non-bis-allylic sites and primarily at the mono-allylic sites. In general, deuteration at the 3 bis-allylic methylene sites converts the 6 hydrogen atoms to deuterium with greater than a 90% efficiency—meaning that in a population of deuterated arachidonic acid, the 6 bis-allylic hydrogen atoms have on average greater than 5.4 deuterium atoms. In addition, no more than about 35% of the total number of hydrogen atoms in arachidonic acid (excluding any ester portion) are replaced with deuterium. Since there are 32 hydrogen atoms in arachidonic acid, the percent of deuterium in these compounds ranges from about 15% to no greater than 35%. It is understood that when the defined term “deuterated arachidonic acid” recites D6 arachidonic acid, such includes arachidonic acid having greater than 5.4 deuterium atoms on average at the bis-allylic sites and a range of deuteration of from about 15% to no greater than 35% based on the number of hydrogen atoms present on non-deuterated arachidonic acid including the proton on the carboxylic acid.

Esters of these deuterated fatty acids are prepared by conventional techniques well known in the art. Such esters preferably are derived from C₁-C₅ alkyl alcohols.

### Methodology—Deuterated Linoleic Acid

Some of the methods described herein utilize the enzymatic conversion of deuterated linoleic acid or an ester thereof to provide for deuterated arachidonic acid. Specifically, it is well known that a portion of the linoleic acid or an ester thereof consumed by an individual is converted to arachidonic acid in vivo, whereby that portion of linoleic acid or ester thereof acts as a prodrug of arachidonic acid.

In one embodiment, a patient treated with 11,11-D2-linoleic acid or ester thereof will generate 13,13-D2-arachidonic acid in vivo. As per above, when the concentration of said 13,13-D2-arachidonic acid in red blood cells reaches at least about 3% based on the total number of arachidonic acid+deuterated arachidonic acid found therein, that concentration is deemed to be therapeutic.

In one embodiment, a patient treated with 8,8,11,11-D4-linoleic acid or ester thereof will generated 10,10,13,13-D4-arachidonic acid in vivo. As per the above, when the concentration of said 10,10,13,13-D4-arachidonic acid in red blood cells reaches at least about 1% based on the total number of arachidonic acid+deuterated arachidonic acid found therein, that concentration is deemed to be therapeutic.

In one embodiment, when the concentration of D6-arachidonic acid in red blood cells reaches at least about 1% based on the total number arachidonic acid+deuterated arachidonic acid found therein, that concentration is deemed to be therapeutic.

It is surprising that each of the treatable neurodegenerative diseases require an equivalent threshold of deuterated arachidonic acid for achieving a minimum therapeutic required concentration for achieving therapeutic results given that each disease has a separate etiology and, in many cases, a separate pathology.

In one embodiment, deuterated linoleic acid, e.g. 11,11-D2-linoleic acid, is administered with a dosing regimen that comprises a primer dose and a maintenance dose. In an embodiment, the primer dose comprises periodic administration of 11,11-D2-linoleic acid or an ester thereof. In an embodiment, the primer dose comprises at least about 7 grams of 11,11-D2-linoleic acid or an ester thereof per day. In an embodiment, the primer dose is continued for at least about 30 days, e.g., to rapidly achieve a therapeutic concentration of 13,13-D2-arachidonic acid in vivo due to enzymatic conversion of a portion of said 11,11-D2-linoleic acid to 13,13-D2-arachidonic acid. In an embodiment, after completion of the primer dose, the maintenance dose is periodically administered and wherein the maintenance dose is at least about 3 grams per day of 11,11-D2-linoleic acid or an ester thereof per day.

### Deuterated Arachidonic Acid

Some of the methods described herein utilize deuterated arachidonic acid or an ester thereof. Such methods preferably employ D2-arachidonic acid, D4-arachidonic acid or D6-arachidonic acid as the particular deuterated arachidonic acid. In the case of D6-arachidonic acid, this includes deuteration generated by catalytic processes as described above. Regardless, the concentration in red blood cells of deuterated arachidonic acid found to be therapeutic is set forth in Table 1 above.

In one embodiment, deuterated arachidonic acid, e.g., 7,7,10,10,13,13-D6-arachidonic acid, is administered with a dosing regimen that comprises a primer dose and a maintenance dose. In an embodiment, the primer dose comprises periodic administration of 7,7,10,10,13,13-D6-arachidonic acid or an ester thereof. In an embodiment, the primer dose comprises about 0.5 grams to about 5 grams of 7,7,10,10,13,13-D6-arachidonic acid or an ester thereof per day. In an embodiment, the primer dose is continued for about 24 days to about 45 days, e.g., to rapidly achieve a therapeutic concentration of 7,7,10,10,13,13-D6-arachidonic acid in vivo. In an embodiment, after completion of the primer dose, the maintenance dose is periodically administered. In an embodiment no more than about 70% of the primer dose of 7,7,10,10,13,13-D6-arachidonic acid or an ester thereof per day is administered.

### SPECIFIC EMBODIMENTS

Embodiment 1. A non-invasive method for determining a concentration of deuterated arachidonic acid in reporter cells of a population of patients that correlates to onset of a therapeutic effect in a patient population suffering from a neurodegenerative disease treatable with said deuterated arachidonic acid, the method comprising:
a) obtaining data from the population of patients having a first neurodegenerative disease during treatment regimen with deuterated arachidonic acid or a prodrug thereof, which data comprises the concentration of deuterated arachidonic acid in the population of patients' reporter cells at one or more times after the start of therapy;
b) assessing when the patients evidence a therapeutic result;
c) identifying the average time from initiation of therapy to onset of therapeutic results and the average concentration of deuterated arachidonic acid in the reporter cells of each patient at that time of such onset for each neurodegenerative disease;
d) correlating the average concentration of the deuterated arachidonic acid in said population of patients' reporter cells to the average time for the onset of a minimum required therapeutic result for said neurodegenerative disease as evidencing that patient neurons have a minimum required therapeutic concentration of the deuterated arachidonic acid for that disease;
e) repeating steps a)-d) for one or more further neurodegenerative diseases;
f) evaluating a minimum required therapeutic concentration of deuterated arachidonic acid and average time for onset of therapeutic results for each of said one or more further neurodegenerative diseases; and
f) obtaining an average minimum required therapeutic concentration and average time for the onset of said therapeutic results for a set of neurodegenerative diseases.

Embodiment 2. A method for determining the response of a patient to the administration of a composition comprising deuterated arachidonic acid or a prodrug thereof during the treatment for a treatable neurodegenerative disease, said method comprises:
obtaining a population of red blood cells from said patient suffering from a neurodegenerative disease at a set point in time after the start of therapy predetermined to be when a therapeutic result should be evident for that disease;
assessing the concentration of said deuterated arachidonic acid in said red blood cells at that time wherein said concentration is relative to the amount of arachidonic acid present including deuterated arachidonic acid;
comparing said assessed concentration to said predetermined concentration as to when a therapeutic result is evident;
determining if said assessed concentration is therapeutic or sub-therapeutic for treatable neurodegenerative diseases; and
optionally altering the dose of said deuterated arachidonic acid or prodrug thereof to said patient based on the said determination.

Embodiment 3. Any of the previous embodiments wherein, the sample of reporter cells is obtained about 1 month after the start of therapy.

Embodiment 4. Any of the previous embodiments wherein the further samples of reporter cells are obtained at intervals of 1 month thereafter, 3 months thereafter, semi-annually thereafter, or annually thereafter.

Embodiment 5. Any of the previous embodiments wherein the reporter cells are red blood cells, skin cells, fat cells, biopsied cells, or epithelial cells.

Embodiment 6. Any of the previous embodiments wherein said deuterated arachidonic acid is 13,13-D2-arachidonic acid

Embodiment 7. Any of the previous embodiments wherein said deuterated arachidonic acid is D4-arachidonic acid.

Embodiment 8. Any of the previous embodiments wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

Embodiment 9. A method for assessing the response of a patient to the administration of a composition comprising deuterated arachidonic acid or a prodrug thereof during the treatment of a treatable neurodegenerative disease, said method comprises:
assessing the concentration of deuterated arachidonic acid in the patient's red blood cells relative to the total amount of arachidonic acid present, including deuterated arachidonic acid;
comparing that concentration to the predetermined minimum required therapeutic concentration for said deuterated arachidonic acid in said red blood cells;
ascribing a sub-therapeutic concentration when the patient's red blood cells have a concentration of said deuterated arachidonic acid which is less than said determined therapeutic concentration and ascribing a therapeutic concentration of said deuterated arachidonic acid when the concentration of said deuterated arachidonic acid is equal to or greater than said determined therapeutic concentration; and
optionally repeating the method as needed to assess whether the patient's concentration of deuterated arachidonic acid in said red blood cells has achieved or is maintaining a therapeutic concentration.

Embodiments 10. Embodiment 9, wherein the sample of reporter cells is obtained about 1 month after the start of therapy.

Embodiment 11. Any of the previous embodiments wherein the further samples of reporter cells are obtained at intervals of 1 month thereafter, 3 months thereafter, semi-annually thereafter, or annually thereafter.

Embodiment 12. Any of the previous embodiments wherein the reporter cells are red blood cells, skin cells, fat cells, biopsied cells, or epithelial cells.

Embodiment 13. Any of the previous embodiments wherein said deuterated arachidonic acid is 13,13-D2-arachidonic acid

Embodiment 14. Any of the previous embodiments wherein said deuterated arachidonic acid is D4-arachidonic acid.

Embodiment 15. Any of the previous embodiments wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

Embodiment 16. A diagnostic method to determine whether the concentration of deuterated 13,13-D2-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with 13,13-D2-arachidonic acid or an ester thereof;
determining the concentration of 13,13-D2-arachidonic acid in red blood cells (RBCs); and
comparing that concentration to a minimum therapeutic concentration of least 3% of 13,13-D2-arachidonic acid based on the total amount of arachidonic acid present in said RBCs including deuterated arachidonic acid to determine if the patient has a therapeutic concentration or a sub-therapeutic of 13,13-D2-arachidonic acid in the neurons.

Embodiments 17. Embodiment 16, wherein said therapeutic concentration of 13,13-D2-arachidonic acid is set at 4%, 5%, 6%, 7%, 8%, 9% or even 10% as the therapeutic target for a given patient or cohort of patients.

Embodiment 18. Any of the previous embodiments wherein the sample of reporter cells is obtained about 1 month after the start of therapy.

Embodiment 19. Any of the previous embodiments wherein the further samples of reporter cells are obtained at intervals of 1 month thereafter, 3 months thereafter, semi-annually thereafter, or annually thereafter.

Embodiment 20. A diagnostic method to determine whether the concentration of deuterated D4-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with D4-arachidonic acid or an ester thereof; and
determining the concentration of D4-arachidonic acid in red blood cells (RBCs); and
comparing that concentration to a minimum therapeutic concentration of least 1% of D4-arachidonic acid based on the total amount of fatty acids in said RBCs including the D4-arachidonic acid to determine if the patient has a therapeutic concentration or a sub-therapeutic of D4-arachidonic acid in the neurons.

Embodiment 21. Embodiment 20 wherein said therapeutic concentration of D4-arachidonic acid is set at 2%, 3%, or 5% as the therapeutic target for a given patient or cohort of patients.

Embodiment 22. Any of the previous embodiments wherein the sample of reporter cells is obtained about 1 month after the start of therapy.

Embodiment 23. Any of the previous embodiments wherein the further samples of reporter cells are obtained at intervals of 1 month thereafter, 3 months thereafter, semi-annually thereafter, or annually thereafter.

Embodiment 24. A diagnostic method to determine whether the concentration of deuterated D6-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with D4-arachidonic acid or an ester thereof; and
determining the concentration of D6-arachidonic acid in red blood cells (RBCs); and
comparing that concentration to a minimum therapeutic concentration of least 0.5% of D6-arachidonic acid based on the total amount of arachidonic acid, including deuterated arachidonic acid in said RBCs to determine if the patient has a therapeutic concentration or a sub-therapeutic of D6-arachidonic acid in the neurons.

Embodiment 25. Embodiments 24 wherein said therapeutic concentration of D6-arachidonic acid is set at 1%, 1.5%, 2%, 2.5%, or 3% as the therapeutic target for a given patient or cohort of patients.

Embodiment 26. Any of the previous embodiments wherein the sample of reporter cells is obtained about 1 month after the start of therapy.

Embodiment 27. Any of the previous embodiments wherein the further samples of reporter cells are obtained at intervals of 1 month thereafter, 3 months thereafter, semi-annually thereafter, or annually thereafter.

Embodiment 28. Any of the previous embodiments wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

Embodiment 29. Any of the previous embodiments placed into a kit of parts which includes diagnostic materials for conducting said methods as well as a correlation table that correlates the concentration of the deuterated arachidonic acid in the red blood cells to that of the cerebral spinal fluid in humans. In addition to the correlation table, such diagnostic materials can include one or more instructions for when such tests should be conducted, factors that suggest delaying such tests, and the like.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.
D2-AA=13,13-D2-Arachidonic Acid
AA=Arachidonic Acid
ALSFRS-R=Revised ALS Functional Rating Scale
BID=Twice a day
CNS=Central Nervous System
D2-LA=11,11-D2-Linoleic Acid
LA=Linoleic Acid
PK=Pharmacokinetics
PSP=Progressive Supranuclear Palsy
RBC=Red Blood Cells
SAE=Serious Adverse Events
SF=Cerebral Spinal Fluid
TID=Three times a day
% deuterated AA=Percentage of deuterated arachidonic acid is based on the total amount of arachidonic acid present in the cell being analyzed including the deuterated arachidonic acid

### Example 1—Determination of AA Concentrations in RBCs and Spinal Fluid/Neurons in a Single Patient

This example determines the relative concentration of D2-AA in the SF and in RBCs in order to determine if there is a 1:1 correlation between these two concentrations based on the relative amount of D2-AA to D2-LA. Specifically, a patient was continuously provided with a daily dose of 9 grams of D2-LA ethyl ester over about a six-month period. Periodic samples of blood and SF were taken and the concentration of both D2-LA and D-2AA in both the RBCs and the SF were measured. In all cases, the D2-AA was obtained by deacylation of the ethyl ester of linoleic acid in the gastrointestinal tract followed by conversion of D2-LA in vivo to D2-AA.

TABLE 2

| | Concentration | Concentration | Ratio of |
|---|---|---|---|
| | of D2-LA | of D2-AA | D2-LA to |
| Time | in SF | in SF | D2-AA in SF |
| 1 month | 19.8% | 8% | 2.5:1 |

The results found in Table show that the concentration of D2-AA in the cerebral spinal fluid is already 8% based on the amount of arachidonic acid+deuterated arachidonic acid.

Next, Table 3 shows that the concentration of D2-LA and D2-AA in the RBCs at 3 months and 6 months for the same patient.

TABLE 3

| | Concentration | Concentration | Ratio of |
|---|---|---|---|
| | of D2-LA | of D2-AA | D2-LA to |
| Time | in RBCs | in RBCs | D2-AA in RBCs |
| 3 months | 34.7% | 11.8% | 2.9:1 |
| 6 months | 34.5 | 16.7 | 2.1:1 |

Note here that the concentration of D2-AA in RBC's at 3 months is significantly less than that at 6 months evidencing the incremental increase in D2-AA over time. Moreover, the ratio of D2-LA to D2-AA changes from 2.9:1 at 3 month to 2.1:1 at 6 months.

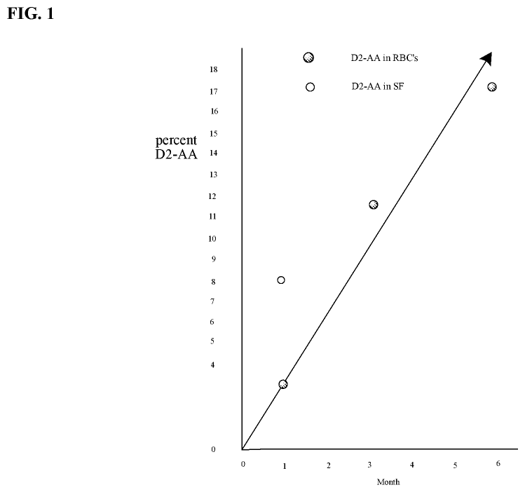

Since the amount of D2-AA is increasing over time in an incremental fashion based on the conversion of D2-LA that limits the amount of D2-AA bio-generated per day, one can assume a fairly linear rate of increase. This is shown in FIG. 1 where the solid line is set by the concentrations of D2-AA at 3 months and 6 months and then extrapolated back to start of therapy (0 months). The value for the D2-AA in RBC's at 1 month is estimated from this linear relationship. The amount shown for 1 month in the SF is also provided (open circle).

Based on the above and FIG. 1, one can see that the data to date suggests that the amount D2-AA at 1 month in RBCs would be about 3 percent as compared to 8% for the amount of D2-AA in the SF. Accordingly, this data suggests that the concentration the body shunts more of the AA (including D2-AA) into the SF (and hence the neurons) as compared to RBCs and likely other reporter cells.

### Example 2—Determination of AA Concentrations in RBCs and Spinal Fluid/Neurons in a Cohort of 14 Patients

This example also determines the concentration of D2-AA in RBCs. Specifically, a cohort of 14 children was provided with a daily dose of 3 grams of D2-LA ethyl ester for months followed by 2 grams of D2-LA ethyl ester for the remaining six-month period. Blood samples were taken at 3 months for all but 1 child and at 6 months for all children. The concentration of D2-AA in RBCs was measured. In all cases, the D2-AA was obtained by deacylation of the ethyl ester of linoleic acid in the gastrointestinal tract followed by conversion of D2-LA in vivo to D2-AA.

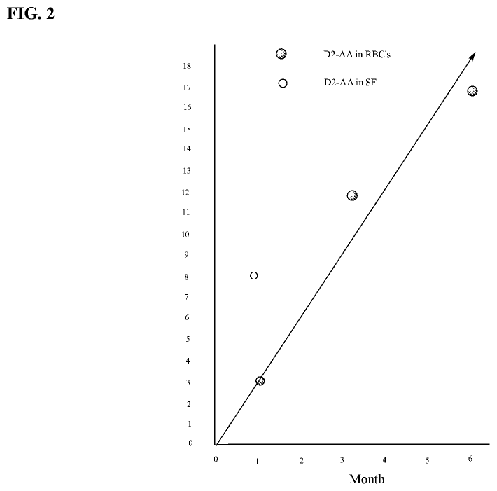

At 3 months, the average concentration of D2-AA in the RBCs was determined to be 12% (6.8% low and 16.8% high). At 6 months, the average concentration of D2-AA in the RBCs was determined to be 16.7% (12.0% low and 26.1% high). FIG. 2 depicts these results assuming a linear relationship of D2-AA accumulation in the body. Included in FIG. 2 is the 1-month data for D2-AA in the spinal fluid as found in Example 1.

As can be seen, FIGS. 1 and 2 are substantially the same strongly suggesting that the dosing of D2-LA to the adult patient in Example 1 and to the children in Example 2 maximized the conversion of D2-LA to D2-AA. This data further suggests that once maximized, the amounts of D2-AA generated over time are reproducible.

### Example 3—Ascertaining a Therapeutic Concentration in ALS

Patients suffering from ALS were treated with D2-LA over a period of time. The patients were given different dosing amounts of D2-LA and for different dosing periods. All patient evaluated had their ALSFRS-R analysis of their natural history prior to start of therapy.

Blood samples from these patients were evaluated for the concentration of D2-AA in their RBCs. PK sampling in 10 patients gave an average D2-AA concentration of 3.1%±2.0 in RBCs. In addition, functional scores for each of the patients (ALSFRS-R results) at the end of therapy were compared to the natural history scores at the start of therapy. Based on this comparison, the rate of decline changed from an annualized rate of −14.2+/−4.4 per year pre-treatment to −7.6+/−1.4 during treatment or 46% reduction (p=0.07, paired t-test for within-subject change in slope).

The results of this study evidence a concentration of D2-AA in RBCs of about 3% based on the total amount arachidonic acid+deuterated arachidonic acid found therein, that concentration is deemed to be therapeutic found therein (including the D2-AA) provides for a therapeutic benefit in reducing the rate of disease progression when the D2-AA concentration in the RBCs is at least about 3 percent.

### Example 4—Ascertaining a Therapeutic Concentration in PSP

Three patients diagnosed with likely PSP (an example of tauopathy) underwent baseline assessment using the 28-item Progressive Supranuclear Palsy Rating Scale (PSPRS) [19] and the Unified Parkinson's Disease Rating Scale (UPDRS). They were then treated with D2-Lin (2.88 g BID; 5.76 g total daily dose) and observed for disease progression. During the treatment period, scores in the 2 rating scales were determined every 3 months. Pharmacokinetic (PK) sampling was performed at month 3. These analytes included plasma and RBC membrane levels of (D2-LA) and its centrally active metabolite D2-AA.

The three patients were 2 males (age 66 and 73) and one female (age 74) each of whom had pre-treatment symptom duration of 6 years and 3 years for the two males and 2 years for the female. The baseline PSPRS for the two males was 17 and 12 respectively and 13 for the female. The baseline UPDRS for the two males was 44 and 36 respectively and 21 for the female.

After 3 months of therapy, the slope of the PSPRS changed from the historical decline of 0.91 points/month to a mean of decline of 0.16 points/month (+/−0.23 SEM). The UPDRS slope changed from an expected increase of 0.95 points/month to an average increase in score of 0.28 points/month (+/−0.41 SEM).

At 3 months, the following data was collected:

| | % ratio of 13,13-D2-AA |
|---|---|
| Patient | to AA + 13,13-D2-AA |
| 1* | 5.9% |
| 2 | 3.3% |
| 3 | 5% |

*patient 1 was evaluated at 4 months

At 12 months, the following data was collected:

| | % ratio of 13,13-D2-AA |
|---|---|
| Patient | to AA + 13,13-D2-AA |
| 1** | 10.5% |
| 2 | 6.9% |
| 3 | 8.6% |

**patient 1 was evaluated at 13 months

The second male patient had his dose increased (2.88 g TID; 8.64 g total daily dose) after the first year of treatment so as to further enhance his therapy.

The above results evidenced that a percent ratio of 13,13-D2-AA to (AA+13,13-D2) in red blood cells of about 3% was necessary to achieve therapeutic results. This data further evidenced that a percent ratio of 13,13-D2-AA to (AA+13,13-D2-AA) in red blood cells of about 5% is preferred; and that 6% or 8% is more preferred.

What is claimed is:

1. A non-invasive method for determining a concentration of deuterated arachidonic acid in reporter cells of a population of patients that correlates to onset of a therapeutic effect in a patient population suffering from a neurodegenerative disease treatable with said deuterated arachidonic acid, the method comprising:
a) obtaining data from the population of patients having a first neurodegenerative disease during treatment regimen with deuterated arachidonic acid or a prodrug thereof, which data comprises the concentration of deuterated arachidonic acid in the population of patients' reporter cells at one or more times after the start of therapy;
b) assessing when the patients evidence a therapeutic result;
c) identifying the average time from initiation of therapy to onset of therapeutic results and the average concentration of deuterated arachidonic acid in the reporter cells of each patient at that time of such onset for each neurodegenerative disease;
d) correlating the average concentration of the deuterated arachidonic acid in said population of patients' reporter cells to the average time for the onset of a minimum required therapeutic result for said neurodegenerative disease as evidencing that patient neurons have a minimum required therapeutic concentration of the deuterated arachidonic acid for that disease;
e) repeating steps a)-d) for one or more further neurodegenerative diseases;
f) evaluating a minimum required therapeutic concentration of deuterated arachidonic acid and average time for onset of therapeutic results for each of said one or more further neurodegenerative diseases; and
f) obtaining an average minimum required therapeutic concentration and average time for the onset of said therapeutic results for a set of neurodegenerative diseases.

2. A method for determining the response of a patient to the administration of a composition comprising deuterated arachidonic acid or a prodrug thereof during the treatment for a treatable neurodegenerative disease, said method comprises:
obtaining a population of red blood cells from said patient suffering from a neurodegenerative disease at a set point in time after the start of therapy predetermined to be when a therapeutic result should be evident for that disease;
assessing the concentration of said deuterated arachidonic acid in said red blood cells at that time wherein said concentration is relative to the amount of arachidonic acid present including deuterated arachidonic acid;
comparing said assessed concentration to said predetermined concentration as to when a therapeutic result is evident;
determining if said assessed concentration is therapeutic or sub-therapeutic for treatable neurodegenerative diseases; and
optionally altering the dose of said deuterated arachidonic acid or prodrug thereof to said patient based on the said determination.

3. The method of claim 1, wherein the sample of reporter cells is obtained about 1 month after the start of therapy.

4. The method of claim 3, wherein the further samples of reporter cells are obtained at intervals of 1 month thereafter, 3 months thereafter, semi-annually thereafter, or annually thereafter.

5. The method of any one of claim 1, 3 or 4 wherein the reporter cells are red blood cells, skin cells, fat cells, biopsied cells, or epithelial cells.

6. The method of claim 2 or 5, wherein said deuterated arachidonic acid is 13,13-D2-arachidonic acid

7. The method of claim 2 or 5, wherein said deuterated arachidonic acid is D4-arachidonic acid.

8. The method of claim 2 or 5, wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

9. A method for assessing the response of a patient to the administration of a composition comprising deuterated arachidonic acid or a prodrug thereof during the treatment of a treatable neurodegenerative disease, said method comprises:
assessing the concentration of deuterated arachidonic acid in the patient's red blood cells relative to the total amount of arachidonic acid present, including deuterated arachidonic acid;
comparing that concentration to the predetermined minimum required therapeutic concentration for said deuterated arachidonic acid in said red blood cells;
ascribing a sub-therapeutic concentration when the patient's red blood cells have a concentration of said deuterated arachidonic acid which is less than said determined therapeutic concentration and ascribing a therapeutic concentration of said deuterated arachidonic acid when the concentration of said deuterated arachidonic acid is equal to or greater than said determined therapeutic concentration; and
optionally repeating the method as needed to assess whether the patient's concentration of deuterated arachidonic acid in said red blood cells has achieved or is maintaining a therapeutic concentration.

10. The method of claim 9, wherein the sample of reporter cells is obtained about 1 month after the start of therapy.

11. The method of claim 10, wherein the further samples of reporter cells are obtained at intervals of 1 month thereafter, 3 months thereafter, semi-annually thereafter, or annually thereafter.

12. The method of any one of claim 9, 10 or 11 wherein the reporter cells are red blood cells, skin cells, fat cells, biopsied cells, or epithelial cells.

13. The method of claim 9, wherein said deuterated arachidonic acid is 13,13-D2-arachidonic acid

14. The method of claim 9, wherein said deuterated arachidonic acid is D4-arachidonic acid.

15. The method of claim 9, wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

16. A diagnostic method to determine whether the concentration of deuterated 13,13-D2-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with 13,13-D2-arachidonic acid or an ester thereof;
determining the concentration of 13,13-D2-arachidonic acid in red blood cells (RBCs); and
comparing that concentration to a minimum therapeutic concentration of least 3% of 13,13-D2-arachidonic acid based on the total amount of arachidonic acid present in said RBCs including deuterated arachidonic acid to determine if the patient has a therapeutic concentration or a sub-therapeutic of 13,13-D2-arachidonic acid in the neurons.

17. The method of claim 16, wherein said therapeutic concentration of 13,13-D2-arachidonic acid is set at 4%, 5%, 6%, 7%, 8%, 9% or even 10% as the therapeutic target for a given patient or cohort of patients.

18. The method of claim 16, wherein the sample of reporter cells is obtained about 1 month after the start of therapy.

19. The method of claim 16, wherein the further samples of reporter cells are obtained at intervals of 1 month thereafter, 3 months thereafter, semi-annually thereafter, or annually thereafter.

20. A diagnostic method to determine whether the concentration of deuterated D4-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with D4-arachidonic acid or an ester thereof; and
determining the concentration of D4-arachidonic acid in red blood cells (RBCs); and comparing that concentration to a minimum therapeutic concentration of least 1% of D4-arachidonic acid based on the total amount of fatty acids in said RBCs including the D4-arachidonic acid to determine if the patient has a therapeutic concentration or a sub-therapeutic of D4-arachidonic acid in the neurons.

21. The method of claim 20, wherein said therapeutic concentration of D4-arachidonic acid is set at 2%, 3%, or 5% as the therapeutic target for a given patient or cohort of patients.

22. The method of claim 20, wherein the sample of reporter cells is obtained about 1 month after the start of therapy.

23. The method of claim 20, wherein the further samples of reporter cells are obtained at intervals of 1 month thereafter, 3 months thereafter, semi-annually thereafter, or annually thereafter.

24. A diagnostic method to determine whether the concentration of deuterated D6-arachidonic acid in a patient with a treatable neurodegenerative disease is therapeutic or sub-therapeutic without accessing the patient's neurons or cerebral spinal fluid, which method comprises:
obtaining a blood sample from a patient being treated for a neurodegenerative disease with D4-arachidonic acid or an ester thereof; and
determining the concentration of D6-arachidonic acid in red blood cells (RBCs); and
comparing that concentration to a minimum therapeutic concentration of least 0.5% of D6-arachidonic acid based on the total amount of arachidonic acid, including deuterated arachidonic acid in said RBCs to determine if the patient has a therapeutic concentration or a sub-therapeutic of D6-arachidonic acid in the neurons.

25. The method of claim 24, wherein said therapeutic concentration of D6-arachidonic acid is set at 1%, 1.5%, 2%, 2.5%, or 3% as the therapeutic target for a given patient or cohort of patients.

26. The method of claim 24, wherein the sample of reporter cells is obtained about 1 month after the start of therapy.

27. The method of claim 24, wherein the further samples of reporter cells are obtained at intervals of 1 month thereafter, 3 months thereafter, semi-annually thereafter, or annually thereafter.

28. The method of claim 24, wherein said deuterated arachidonic acid is D6-arachidonic acid wherein said deuterated D6-arachidonic acid comprises at least 90% of the hydrogen atoms at the 7,7,10,10,13,13 positions replaced with deuterium and optionally having up to 35% of the remaining hydrogen atoms at the non-bis-allylic sites replaced by deuterium.

29. A kit of parts which includes diagnostic materials for conducting said methods as well as a correlation table that correlates the concentration of the deuterated arachidonic acid in the red blood cells to that of the cerebral spinal fluid in humans. In addition to the correlation table, such diagnostic materials can include one or more instructions for when such tests should be conducted, factors that suggest delaying such tests, and the like.
